(19) United States
(12) Patent Application Publication
(10) Pub. No.: US 2014/0329781 A1
(43) Pub. Date: Nov. 6, 2014

(54) METHODS FOR TREATING CARDIOVASCULAR DISORDER

(71) Applicant: WOCKHARDT LIMITED, Aurangabad (IN)

(72) Inventors: Mandar Kodgule, Mumbai (IN); Amit Gupta, Uttar Pra-desh (IN); Premchand Nakhat, Yavatmal (IN); Girish Kumar Jain, Delhi (IN)

(73) Assignee: WOCKHARDT LIMITED, Aurangabad (IN)

(21) Appl. No.: 14/298,898

(22) PCT Filed: Nov. 1, 2012
(86) PCT No.: PCT/IB2012/056084

§ 371 Date: Jun. 7, 2014

(30) Foreign Application Priority Data
Dec. 9, 2011 (IN) 3481/MUM/2011

(51) Int. Cl. A61K 31/616; A61K 31/4365; A61K 31/519; A61K 31/138

(52) U.S. Cl. CPC A61K 31/616; A61K 31/138; A61K 31/4365; A61K 31/519
USPC 514/161; 514/301; 514/261.1; 514/162

(57) ABSTRACT

The present invention relates to a once-a-day therapeutically synergistic pharmaceutical dosage form for treatment of cardiovascular disorders, wherein the dosage form comprises a fixed dose combination of metoprolol in extended release form and one or more antiplatelet agent along with one or more rate controlling excipients.

### FIELD OF THE INVENTION

The present invention relates to a once-a-day therapeutically synergistic pharmaceutical dosage form for treatment of cardiovascular disorders, wherein the dosage form comprises a fixed dose combination of metoprolol in extended release form and one or more antiplatelet agent along with one or more rate controlling excipients.

### BACKGROUND OF THE INVENTION

“Cardiovascular disease or disorder” is intended to mean any cardiovascular disease or disorder known in the art, including, but not limited to, congestive heart failure, complications associated with diabetes mellitus, hyperhomocysteinemia, hypercholesterolemia, atherosclerosis, inflammatory heart disease, valvular heart disease, restenosis, hypertension (e.g. pulmonary hypertension, labile hypertension, idiopathic hypertension, low-renin hypertension, salt-sensitive hypertension, low-renin, salt-sensitive hypertension, thromboembolic pulmonary hypertension; pregnancy-induced hypertension; renovascular hypertension; hypertension-dependent end-stage renal disease, hypertension associated with cardiovascular surgical procedures, hypertension with left ventricular hypertrophy, and the like), diastolic dysfunction, coronary artery disease, myocardial infarctions, cerebral infarctions, arteriosclerosis, atherogenesis, cerebrovascular disease, angina (including chronic, stable, unstable and variant (Prinzmetal) angina pectoris), aneurysm, ischemic heart disease, cerebral ischemia, myocardial ischemia, thrombosis, platelet aggregation, platelet adhesion, smooth muscle cell proliferation, vascular or non-vascular complications associated with the use of medical devices, vascular or non-vascular wall damage, peripheral vascular disease, neointimal hyperplasia following percutaneous transluminal coronary angiograph, vascular grafting, coronary artery bypass surgery, thromboembolic events, post-angioplasty restenosis, coronary plaque inflammation, embolism, stroke, shock, arrhythmia, atrial fibrillation or atrial flutter, thrombotic occlusion and reclusion cerebrovascular incidents, and the like.

Many individuals at an elevated risk of suffering serious to life-threatening cardiovascular events, such as myocardial infarction (heart attack), cardiac arrest, congestive heart failure, stroke, peripheral vascular disease and/or claudication. And the risk factors for these are numerous and widespread throughout the world. They include cigarette smoking, diabetes, hypercholesterolemia (high serum cholesterol), hypertension, angina, systemic lupus erythematosus, prior heart attacks or strokes, hemodialysis, hyperhomocysteine levels, obesity, sedentary lifestyle, receiving an organ transplant, atherosclerosis, and others. There is a need for a safe and convenient pharmaceutical formulation that would effectively reduce the risk of incurring a cardiovascular event in individuals who have these risk factors.

Many individuals at an elevated risk of suffering serious to life-threatening cardiovascular events, such as myocardial infarction (heart attack), cardiac arrest, congestive heart failure, stroke, peripheral vascular disease and/or claudication. And the risk factors for these are numerous and widespread throughout the world. They include cigarette smoking, diabetes, hypercholesterolemia (high serum cholesterol), hypertension, angina, systemic lupus erythematosus, prior heart attacks or strokes, hemodialysis, hyperhomocysteine levels, obesity, sedentary lifestyle, receiving an organ transplant, atherosclerosis, and others. There is a need for a safe and convenient pharmaceutical formulation that would effectively reduce the risk of incurring a cardiovascular event in individuals who have these risk factors.

The treatments and drugs discovered or known in the art for cardiovascular disease includes the beta-blockers, for example, atenolol, metoprolol, nadolol, oxprenolol, pindolol, propranolol, timolol; Alpha blockers, for example, doxazosin, phentolamine, indoramin, phenoxybenzamine, prazosin, terazosin, tolazoline; mixed alpha and beta blockers, for example, bucindolol, carvedilol and labetalol, etc.

Beta-blocker, for example, metoprolol acts by blocking the adrenergic stimulation of the heart and thus reduces the oxygen demand of the cardiac tissue. Apparently, this explains their beneficial effects in angina pectoris and cardioprotective action in myocardial infarction. In addition, beta-blockers normalize blood pressure in a large proportion of patients with arterial hypertension, which probably is due to an additional action on the control of peripheral resistance to blood-flow.

Metoprolol (Formula I) is a beta1-selective (cardioselective) adrenoreceptor-blocking agent. It is commercially available in two salt forms; one of them is tartrate salt available as Lopressor tablets and the other is succinate salt available as Toprol-XL tablets. The Toprol XL tablets contain 23.75, 47.5, 95 and 190 mg of metoprolol succinate equivalent to 25, 50, 100 and 200 mg of metoprolol tartrate, USP, respectively. Metoprolol is indicated in the treatment of hypertension, heart failure and angina pectoris.

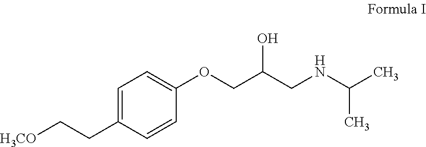 [SMILES: COCCc1ccc(OCC(O)CNC(C)C)cc1]

Initial therapy with a diuretic or beta-blocker has been the usual first approach for treating cardiovascular disorders. Several fixed dose combinations of anti-hypertensive drugs are available in the market. One of the commercially available cardiovascular drug combinations include Lopressor HCT® (Metoprolol and Hydrochlorthiazide).

These cardiovascular combinations or individual drugs are also prescribed along with other drugs such as cardioprotectant, platelet aggregation inhibitors, anticoagulants, etc.

Anti-platelet drugs are class of pharmaceuticals that decrease platelet aggregation and inhibit thrombus formation. They are widely used in primary and secondary prevention of thrombotic cerebrovascular or cardiovascular disease. Commonly used anti-platelet drugs include Cyclooxygenase inhibitors e.g. Aspirin, Adenosine diphosphate (ADP) receptor inhibitors e.g. Clopidogrel (Plavix®); Prasugrel (Effient®); Ticagrelor (Brilinta®); Ticlopidine (Ticlid®); Phosphodiesterase inhibitors e.g. Cilostazol (Pletal®); Adenosine reuptake inhibitors e.g. Dipyridamole (Persantine), etc.

Aspirin suppress the production of prostaglandins and thromboxanes due to its irreversible inactivation of the cyclooxygenase enzyme required for prostaglandin and thromboxane synthesis. Low-dose, long-term aspirin use irreversibly blocks the formation of thromboxane A2 in platelets, producing an inhibitory effect on platelet aggregation. This antithrombotic property makes aspirin useful for reducing the incidence of heart attacks.

Clopidogrel specifically and irreversibly inhibits the P2Y₁₂ subtype of ADP receptor, which is important in aggregation of platelets and cross-linking by the protein fibrin. The blockade of this receptor inhibits platelet aggregation by blocking activation of the glycoprotein IIb/IIIa pathway.

As per the World Heart Federation, hypertension is the single most important risk factor for stroke. It causes about 50 percent of ischemic strokes and also increases the risk of hemorrhagic stroke.

Hypertension places strain on all blood vessels, thus makes them weaken and predisposes them to damage. In such situation heart also work harder to keep blood circulating. Once the blood vessels are gets weaken, they are more likely to block. This can cause an ischemic stroke or transient ischemic attacks. Less often, hypertension is implicated in hemorrhagic strokes when a blood vessel in the brain bursts and blood leaks into the brain.

Hence, for preventing the transient ischemic attacks, there is need for a fixed dose combination comprising anti-hypertensive agents along with one or more antiplatelet agent.

The drawback of using cardiovascular drugs alone, for example metoprolol, is release of some prostaglandins in the body, which may occur shortly after administration of metoprolol dosage. Generally, the production of prostaglandin suppress with the anti-platelet agent such as aspirin, clopidogrel and the like or combination thereof.

Hence, combinations of beta-blockers with anti-platelet agents are expected to provide a better control over various cardiovascular diseases. The said combinations can be given as two separate drugs administered separately at same time or at different timings.

However, in the combination product, each component causes a proportional risk reduction. The long-term benefits would be even larger, perhaps more than a 75 percent overall risk reduction, since risk is only partially reversed in the first one to two years of blood pressure and cholesterol lowering treatment.

The problem associated with these fixed dose combination is that it does not provide physician an option to modulate the dose of drugs within these fixed dose combinations according to need of a patient.

Since cardiovascular disorders are often chronic in nature, complex drug regimen involving several drugs has a negative impact on patient's life leading to non-compliance. Moreover multiple medication administration, complex drug regimen, and frequent dose administration complicates the patient's compliance. Further, it becomes difficult for the physician to prescribe appropriate doses of different drugs when used in combination.

U.S. Pat. Nos. 4,847,265 and 4,529,596 disclose clopidogrel and German Patent No. 218467 discloses aspirin.

Compositions comprising beta-adrenergic blockers and/or antiplatelet agent have been suggested in the prior art.

U.S. Pat. No. 5,156,849 discloses a capsule comprising atenolol, aspirin, and a barrier around the atenolol to prevent interaction between the aspirin and atenolol.

U.S. Patent application No. 2004/0198839A1 discloses a single dosage form of a beta-blocker agents and a platelet aggregation. The application, however, neither discloses conditions for formation of single dosage form nor its release profile, which may be responsible for synergistic therapeutic effect in cardiovascular patient.

U.S. Patent application No. 2010/0261684A1 discloses pharmaceutical combination of aspirin or clopidogrel, metoprolol and nitroglycerin as sub-lingual tablet.

The prior art references do not disclose fixed dose combination of metoprolol in extended release form along and antiplatelet agent and use of such composition for treating cardiovascular disease.

Metoprolol is a cardioselective beta-blocker that has been classified as a class I substance according to the Biopharmaceutics Classification Scheme BCS, meaning that it is highly soluble and highly permeable. The drug is readily and completely absorbed throughout the whole intestinal tract but is subject to extensive first pass metabolism resulting in incomplete bioavailability (about 50%). On the other hand, clopidogrel, a representative example in class of anti-platelet drugs, is poorly water-soluble drug. Clopidogrel is largely protein bound to albumin (94-98%) and undergoes extensive first pass metabolism in the liver with a low bioavailability. Thus, formulating a once-a-day dosage form of highly water soluble metoprolol in a fixed dose combination comprising an extended release metoprolol and poorly water soluble clopidogrel is a challenging task for a pharmacist.

None of the above mentioned prior arts completely provide a once-a-day fixed dose formulation comprising an extended release metoprolol and anti-platelet agent, which is safe and has an enhanced therapeutic effect over the existing individual drug therapy. The combination disclosed in the prior arts also does not address the uniform release and bioavailability related aspects of either metoprolol or antiplatelet agents when formulated into a once-a-day dosage form. Further, preparing a fixed dose combination comprising metoprolol in an extended release dosage form was also a major challenge as it was difficult to achieve the desired therapeutic release of the combination when combined into a single unit dosage form. Therefore, there is the need for the development of new dosage forms comprising an extended release metoprolol and antiplatelet agent, which are safe and effective.

### SUMMARY OF THE INVENTION

In one general aspect of the invention, there is provided a pharmaceutical dosage form for treatment of cardiovascular disorders suitable for once daily administration comprising a fixed dose combination of metoprolol or salt thereof in extended release form and one or more antiplatelet agents or salt thereof along with one or more rate controlling excipients.

In another general aspect of the invention, there is provided a once-a-day pharmaceutical dosage form for treatment of cardiovascular disorders, wherein the dosage form comprises a fixed dose combination of about 25 mg to about 200 mg of metoprolol or salt thereof and about 5 mg to about 770 mg of one or more anti-platelet agents or salt thereof.

In another general aspect of the invention, the once-a-day pharmaceutical dosage form exhibits immediate release of antiplatelet agent.

In another general aspect of the invention, the extended release metoprolol component of the dosage form comprises water swellable or water insoluble inert core coated with one or more rate controlling excipient.

In another general aspect of the invention, the water swellable core comprises microcrystalline cellulose, hydroxypropyl methylcellulose, starch or mixtures thereof.

In another general aspect of the invention, the water insoluble inert core comprises silicon dioxide, glass particles, plastic resin particles or mixtures thereof.

In another general aspect of the invention, the rate controlling excipient comprises one or more polymeric rate controlling excipients, non-polymeric rate controlling excipients, or mixtures thereof.

In another general aspect of the invention, the polymeric rate controlling excipient is selected from the group consisting of one or more of cellulose derivatives; polyhydric alcohols; saccharides, gums and derivatives thereof; vinyl derivatives, polymers, copolymers or mixtures thereof; maleic acid copolymers; polyalkylene oxides or copolymers thereof; acrylic acid polymers and acrylic acid derivatives; or any combinations thereof and non-polymeric rate controlling excipient is selected from the group consisting of fat, wax, fatty acid, fatty acid ester, long chain monohydric alcohol or their ester or any combinations thereof.

In another general aspect of the invention, there is provided a pharmaceutical dosage form for treatment of cardiovascular disorders suitable for once daily administration comprising a fixed dose combination of metoprolol or salt thereof in extended release form and one or more antiplatelet agents or salt thereof along with one or more rate controlling excipients, wherein the composition exhibits a dissolution profile such that less than 6% of metoprolol is released within 1 hour; 30%-50% of metoprolol is released within 6 hours and at least 90% of metoprolol is released after 20 hours when the release rate is measured in Apparatus 2 (USP, Dissolution, paddle, 50 rpm) using 500 ml of pH 6.8 phosphate buffer at 37° C.±0.5° C. as dissolution medium.

In another general aspect of the invention, the pharmaceutical composition comprises pharmaceutically acceptable excipients selected from one or more diluents, binders, glidants, solubilizers, lubricants, disintegrants, colorants, suspending agent, thickeners or taste masking agents.

In another general aspect of the invention, the pharmaceutical dosage form is in the form of a tablet, a capsule, granules, a tablet in tablet, tablet/s in capsule, granules in capsule, an orally disintegrating tablet, a bilayer tablet, a trilayer tablet, an in-lay tablet, or suspension.

In another general aspect of the invention, there is provided a method of treating one or more disorders selected from hypertension, congestive heart failure, angina, myocardial infarction, arteriosclerosis, diabetic nephropathy, diabetic cardiac myopathy, renal insufficiency, peripheral vascular disease, left ventricular hypertrophy, cognitive dysfunction, and chronic heart failure, wherein the method comprises administering a once-a-day pharmaceutical dosage form comprising a fixed dose combination of metoprolol or its salt in extended release form and one or more antiplatelet agents or salt thereof along with one or more rate controlling excipients to a patient in need of said treatment.

Embodiments of the pharmaceutical composition may include one or more of the following features. For example, the pharmaceutically acceptable excipients may include diluents, disintegrants, binders, bulking agents, anti-adherents, anti-oxidants, buffering agents, colorants, flavoring agents, coating agents, plasticizers, stabilizers, preservatives, lubricants, glidants, chelating agents, and the like known to the art used either alone or in combination thereof.

### DETAILED DESCRIPTION OF THE INVENTION

The present inventors while working on the development of pharmaceutical composition comprising a fixed dose combination of an extended release metoprolol with one or more anti-platelet agents, surprisingly found that the pharmaceutical composition of the present invention provides a predictable and uniform dissolution profile resulting in therapeutically effective release of drugs for about 24 hours.

The present invention provides a once-a-day fixed dose therapeutically synergistic pharmaceutical dosage form comprising an extended release metoprolol and immediate release anti-platelet agents, which are not only safe and effective medication for treatment of cardiovascular disease but is also found to be synergistic with enhanced efficacy. This increased efficacy simplifies the management of cardiovascular diseases.

The present inventors have now developed a once-a-day therapeutically synergistic pharmaceutical dosage form comprising an extended release metoprolol, anti-platelet agent and one or more rate controlling excipients, which is safe and effective. From the preliminary studies, applicants have surprisingly found that the combination therapy results in at least 5% reduction in risk of stroke based on individual's response when compared to monotherapy.

The term “metoprolol”, as used herein, refers to a metoprolol base, or any pharmaceutically acceptable salt thereof. For the purpose of present invention metoprolol salt could be metoprolol succinate or tartrate.

In further embodiment, the fixed dosage form of the present invention comprises metoprolol succinate 23.75 mg, 47.5 mg, 95 mg and 190 mg equivalent to 25 mg, 50 mg, 100 mg and 200 mg of metoprolol tartrate or equivalent to 9.75 mg, 19.5 mg, 39 mg and 78 mg of metoprolol base respectively.

The term “anti-platelet agents”, as used herein, refers to anti-platelet drug base, or any pharmaceutically acceptable salt or ester thereof.

As used herein, the term “salt” refers to any pharmaceutically acceptable salt (e.g., acid or base) of a compound of the present invention, which upon administration to a subject is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts. Examples of bases include, but are not limited to, alkali metals (e.g., sodium) hydroxides, alkaline earth metals (e.g., magnesium), hydroxides, ammonia, and compounds of formula NW4+, wherein W is C1-4 alkyl, and the like. Examples of salts include, but are not limited to: acetate, adipate, alginate, aspartate, benzoate, benzenesulfonate, bisulfate, butyrate, citrate, camphorate, camphorsulfonate, digluconate, dodecylsulfate, cyclopentanepropionate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, etc.

As used herein, the term “ester” refers to any pharmaceutically acceptable ester of a compound of the present invention, which upon administration to a subject, is capable of providing a compound of this invention or an active metabolite or residue thereof. Representative examples of ester include medoxomil, cilexetil, etc.

For the purpose of present invention, “once-a-day” means that the composition of the present invention is administered only once over a twenty-four hour period thereby providing therapeutically beneficial blood levels of the drug(s).

The term “fixed dose combination”, as used herein, refers to a combination of two or more separate drug ingredients, combined in a single unit dosage form, in defined doses.

The term “therapeutically synergistic”, as used herein, refers to a therapeutic effect achieved by a fixed dose combination treatment that exceeds the optimal effect achieved by monotherapy associated with the same drugs used in the combination. For example, X is the therapeutic effect obtained by “A” drug and Y is the therapeutic effect obtained by “B” drug on administration, thus when “A” and “B” drugs are given together, then the expected therapeutic effect would be “X+Y” but when the therapeutic effect achieved by co-administration of both the drugs in a fixed dose combination exceeds “X+Y” i.e. “(X+Y)*Z”, wherein Z is more than 1, the combination is said to be therapeutically synergistic.

The phrase “inert core,” as used herein, includes core that is water insoluble and non-swellable.

The phrase “insoluble,” as used herein, refers to inert core, which does not dissolve in water.

The phrase “non-swellable,” as used herein, refers to inert core having 20% or less swelling after 24 hours.

The term ‘inlayed tablet’ as used herein refers to a type of a layered tablet in which instead of the core tablet being completely surrounded by a coating, the top surface is completely exposed.

The term ‘inlayed in said layer’ is used herein to mean that the tablet of metoprolol may be present at any position in said layer.

The term “bioavailable” as used herein, includes, but is not limited to the rate and extent to which the drug(s) become bioavailable to the site of action after administration.

The term “Cmax” is the highest plasma concentration of the drug attained within the dosing interval.

The term “Tmax” is the time period, which elapses after administration of the dosage form at which the plasma concentration of the drug attains the highest plasma concentration of drug attained within the dosing interval.

The term “AUC₀₋ₜ” as used herein, means area under plasma concentration-time curve from drug administration to last observed concentration at time′t′.

The term “AUC_{0-α}” as used herein, means area under the plasma concentration-time curve extrapolated to infinite time.

The term “mean”, when preceding a pharmacokinetic value (e.g. mean Tmax) represents the mean value of the pharmacokinetic value taken from a population of patients or healthy volunteers.

The present invention provides once-a-day therapeutically synergistic pharmaceutical dosage form for treatment of cardiovascular disorders, wherein the dosage form comprises a fixed dose combination of an extended release metoprolol with one or more antiplatelet agents.

In an embodiment, when the once-a-day therapeutically synergistic pharmaceutical dosage form of the present invention comprises metoprolol in extended release form and antiplatelet agents, the amount of metoprolol and antiplatelet agents in the dosage form ranges between about 25 mg to about 200 mg and between about 5 mg to about 770 mg respectively.

In another embodiment, the present invention provides a once-a-day therapeutically synergistic pharmaceutical dosage form for treatment of cardiovascular disorders, wherein a unit dosage form comprises an extended release metoprolol and anti-platelet agent in following combinations:

| Metoprolol | Doses of “Anti-platelet agent” in combination with metoprolol | | | | | | | |
|---|---|---|---|---|---|---|---|---|
| Doses | Clopidogrel | Aspirin | Anagrelide | Dipyridamole | Cilostazol | Ticagrelor | Prasugrel | Ticlopidine |
| 25 mg | 75 mg | 25 mg | 0.5 mg | 25 mg | 5 mg | 90 mg | 5 mg | 250 mg |
| 50 mg | 300 mg | 75 mg | 1 mg | 50 mg | 20 mg | | 10 mg | |
| 100 mg | | 80 mg | | 75 mg | 40 mg | | | |
| 200 mg | | 81 mg | | | | | | |
| | | 300 mg | | | | | | |

In a further embodiment, the present invention provides a pharmaceutical dosage form for treatment of cardiovascular disorders suitable for once daily administration comprising a fixed dose combination of metoprolol or salt thereof in extended release form and one or more antiplatelet agents or salt thereof along with one or more rate controlling excipients, wherein the composition exhibits a dissolution profile such that less than 6% of metoprolol is released within 1 hour; 30%-50% of metoprolol is released within 6 hours and at least 90% of metoprolol is released after 20 hours when the release rate is measured in Apparatus 2 (USP, Dissolution, paddle, 50 rpm) using 500 ml of pH 6.8 phosphate buffer at 37° C.±0.5° C. as dissolution medium.

As mentioned in several embodiments of the present invention, the rate controlling excipient is polymeric rate controlling excipient, non-polymeric rate controlling excipient, or combination thereof.

Suitable polymeric rate controlling excipients are selected from, but not limited to, one or more of cellulose derivatives; polyhydric alcohols; saccharides, gums and derivatives thereof; vinyl derivatives, polymers, copolymers or mixtures thereof; maleic acid copolymers; polyalkylene oxides or copolymers thereof; acrylic acid polymers and acrylic acid derivatives; or any combinations thereof.

Cellulose derivatives include, but are not limited to, ethyl cellulose, methylcellulose, hydroxypropylmethylcellulose (HPMC), hydroxypropyl cellulose (HPC), hydroxyethyl cellulose, hydroxymethyl cellulose, hydroxypropyl ethylcellulose, carboxymethylethylcellulose, carboxyethylcellulose, carboxymethyl hydroxyethylcellulose, hydroxyethyl methyl carboxymethyl cellulose, hydroxyethyl methyl cellulose, carboxymethyl cellulose (CMC), methyl hydroxyethyl cellulose, methylhydroxypropyl cellulose, carboxymethyl sulfoethyl cellulose, sodium carboxymethyl cellulose, or combinations thereof.

Polyhydric alcohols include, but are not limited to, polyethylene glycol (PEG) or polypropylene glycol; or any combinations thereof.

Saccharides, gums and their derivatives include, but are not limited to, dextrin, polydextrin, dextran, pectin and pectin derivatives, alginic acid, sodium alginate, polygalacturonic acid, xylan, arabinoxylan, arabinogalactan, starch, hydroxypropyl starch, amylose and amylopectin, CMC agar; guar gum, locust bean gum, xanthan gum, karaya gum, tragacanth, carrageenan, acacia gum, arabic gum or gellan gum or the like; or any combinations thereof.

Vinyl derivatives, polymers, copolymers or mixtures thereof include, but are not limited to, polyvinyl acetate, polyvinyl alcohol, mixture of polyvinyl acetate (8 parts w/w) and polyvinylpyrrolidone (2 parts w/w) (Kollidon SR), copolymers of vinyl pyrrolidone, vinyl acetate copolymers, polyvinylpyrrolidone (PVP); or combinations thereof.

Polyalkylene oxides or copolymers thereof include, but are not limited to, polyethylene oxide, polypropylene oxide, poly(oxyethylene)-poly (oxypropylene) block copolymers (poloxamers) or combinations thereof.

Maleic acid copolymers include, but are not limited to, vinylacetate-maleic acid anhydride copolymer, styrene-maleic acid anhydride copolymer, styrene-maleic acid monoester copolymer, vinylmethylether-maleic acid anhydride copolymer, ethylene-maleic acid anhydride copolymer, vinylbutyiether-maleic acid anhydride copolymer, acrylonitrile-methyl acrylate-maleic acid anhydride copolymer, butyl acrylate-styrene-maleic acid anhydride copolymer or the like or any combinations thereof.

Acrylic acid polymers include any suitable polyacrylic acid polymers or carboxyvinyl polymers such as those available under the brand name carbopol. Pharmaceutically acceptable acrylic polymer may be include one or more, but not limited to acrylic acid and methacrylic acid copolymers, methyl methacrylate copolymers, ethoxyethyl methacrylates, cynaoethyl methacrylate, aminoalkyl methacrylate copolymer, poly(acrylic acid), poly(methacrylic acid), methacrylic acid alkylamide copolymer, poly(methyl methacrylate), poly(methacrylic acid) (anhydride), methyl methacrylate, polymethacrylate, poly(methyl methacrylate), poly(methyl methacrylate) copolymer, polyacrylamide, aminoalkyl methacrylate copolymer, poly(methacrylic acid anhydride), and glycidyl methacrylate.

Suitable non-polymeric rate controlling excipient includes, but not limited to fat, fat, wax, fatty acid, fatty acid ester, long chain monohydric alcohol or their ester or any combinations thereof.

Waxes are esters of fatty acids with long chain monohydric alcohols. Natural waxes are often mixtures of such esters, and may also contain hydrocarbons. Waxes employed in the present invention include, but are not limited to, natural waxes, such as animal waxes, vegetable waxes, and petroleum waxes, paraffin waxes, microcrystalline waxes, petrolatum waxes, mineral waxes), and synthetic waxes. Specific examples include, but are not limited to spermaceti wax, carnauba wax, Japan wax, bayberry wax, flax wax, beeswax, yellow wax, Chinese wax, shellac wax, lanolin wax, sugarcane wax, candelilla wax, castor wax paraffin wax, microcrystalline wax, petrolatum wax, carbowax, and the like, or mixtures thereof.

Waxes are also monoglyceryl esters, diglyceryl esters, or glyceryl esters (glycerides) and derivatives and mixtures thereof formed from a fatty acid having from about 10 to about 22 carbon atoms and glycerol, wherein one or more of the hydroxyl groups of glycerol are substituted by a fatty acid. Glycerides employed in the present invention include, but are not limited to, glyceryl monostearate, glyceryl distearate, glyceryl tristearate, glyceryl dipalmitate, glyceryl tripalmitate, glyceryl monopalmitate, glyceryl palmitostearate, glyceryl dilaurate, glyceryl trilaurate, glyceryl monolaurate, glyceryl didocosanoate, glyceryl tridocosanoate, glyceryl monodocosanoate, glyceryl monocaproate, glyceryl dicaproate, glyceryl tricaproate, glyceryl monomyristate, glyceryl dimyristate, glyceryl trimyhstate, glyceryl monodecenoate, glyceryl didecenoate, glyceryl tridecenoate, glyceryl behenate (compritol), polyglyceryl diisostearate, lauroyl macrogolglycerides (Gelucire), oleoyl macrogolglycerides, stearoyl macrogolglycerides, mixtures of monoglycerides and diglycerides of oleic acid (Peceol), or combinations thereof.

Fatty acids include, but are not limited to, hydrogenated palm kernel oil, hydrogenated peanut oil, hydrogenated palm oil, hydrogenated rapeseed oil, hydrogenated rice bran oil, hydrogenated soybean oil, hydrogenated sunflower oil, hydrogenated castor oil (Lubritab), hydrogenated cottonseed oil, and mixtures thereof. Other fatty acids include, but are not limited to, decenoic acid, docosanoic acid, stearic acid, palmitic acid, lauric acid, myristic acid, or the like, or mixtures thereof.

Long chain monohydric alcohols include, but are not limited to, cetyl alcohol, or stearyl alcohol or mixtures thereof.

The water-swellable core can comprise hydroxypropyl methylcellulose, microcrystalline cellulose, starch or mixtures thereof.

The water-insoluble inert core can comprise silicon dioxide, small particles of glass, plastic resin particles or mixtures thereof.

The pharmaceutical composition of the present invention further comprises other pharmaceutically acceptable excipient selected from the group consisting of diluent, binder, glidant, solubilizer, stabilizer, lubricants, disintegrants, cushioning agents, suspending agent, thickening agent, sweetners, flavoring agent, or plasticizer.

Examples of suitable diluents include but are not limited to one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, or metal carbonate.

Examples of suitable binders include, but are not limited to, starch, gums, pregelatinized starch, polyvinyl prrolidone (PVP), copovidone, cellulose derivatives, such as hydroxypropylmethyl cellulose (HPMC), hydroxypropyl cellulose (HPC) and carboxymethyl cellulose (CMC) and their salts.

Suitable lubricants include but are not limited to one or more talc, magnesium stearate, calcium stearate, polyethylene glycol, hydrogenated vegetable oils, stearic acid, sodium stearyl fumarate, talc and sodium benzoate.

Compositions of the present invention may include a glidant such as, but not limited to, colloidal silica, silica gel, precipitated silica, or combinations thereof.

Suitable disintegrant may include but are not limited to one or more of starch, croscarmellose sodium, crospovidone, and sodium starch glycolate.

The solubilizer may include but are not limited to one or more surfactant, pH modifier, complexing agent, or hydrotropic agent.

Suitable surfactants are those known to ordinary skilled in the art and may include, but not limited to one or more of amphoteric, non-ionic, cationic or anionic surfactants. Suitable surfactants comprises one or more of sodium lauryl sulfate, monooleate, monolaurate, monopalmitate, monostearate or another ester of polyoxyethylene sorbitane, sodium dioctylsulfosuccinate (DOSS), lecithin, stearylic alcohol, cetostearylic alcohol, cholesterol, polyoxyethylene ricin oil, polyoxyethylene fatty acid glycerides, poloxamer, cremophore RH 40 and the like.

Suitable pH modifiers include but are not limited to buffers, amino acids or amino acid sugars.

The complexing agents include cyclodextrin class of molecules, such as cyclodextrins containing from six to twelve glucose units, especially, alpha-cyclodextrin, beta-cyclodextrin, gamma-cyclodextrin, or their derivatives, such as hydroxypropyl beta cyclodextrins, or mixtures thereof. The complexing agents may also include cyclic amides, hydroxyl benzoic acid derivatives as well as gentistic acid.

Suitable plasticizers include, but are not limited to, one or more of diethyl phthalate, triethyl citrate, acetyl tributyl citrate, dibutyl phthalate, triacetin, propylene glycol, and polyethylene glycol.

The solvents comprise one or more of dichloromethane, acetone, ethanol, methanol, isopropyl alcohol, water or mixture thereof.

Suitable cushioning agents include but are not limited to one or more of PEG, and colloidal silicon dioxide.

Suitable thickening agents or viscosity modifiers may include, but note limited to one or more of methylcellulose, carboxymethylcellulose, microcrystalline cellulose, ethylcellulose, hydroxyethylcellulose, hydroxypropylcellulose, hydroxypropylmethylcellulose, alginate, carageenan, xanthan gum, acacia, tragacanth, locust bean gum, guar gum, carboxypolymethylene, polyvinyl pyrrolidone, polyvinyl alcohol, poloxamer, magnesium aluminum silicate (veegum), bentonite, hectorite, povidone, maltitol, chitosan or combination thereof.

Preservatives may include one or more of sodium benzoate, sorbates, such as potassium sorbate, salts of edetate (also known as salts of ethylenediaminetetraacetic acid or EDTA, such as disodium edetate), benzaldionium chloride, parabens.

The formulations of the invention optionally include one or more stabilizing agents to increase the stability and/or compatibility of the suspension when formulated into a dosage form. Suitable stabilizing agents are suspending agents, flocculating agents, thickening agents, gelling agents, buffering agents, antioxidants, preservatives, antimicrobial agents, and mixtures thereof. Ideally, the agent acts to minimize irreversible aggregation of suspended particles, and to maintain proper flow characteristics to ease manufacturing processes, e.g., to ensure that the formulation can be readily pumped and filled into desired container.

Suspending agents may include, but are not limited to, one or more from cellulose derivatives, clays, natural gums, synthetic gums, or other agents known in the art. Specific suspending agents, by way of example, include microcrystalline cellulose, sodium carboxymethylcellulose, powdered cellulose, ethymethylcellulose, hydroyxypropyl methylcellulose, methylcellulose, ethylcellulose, ethylhydroxy ethylcellulose, hydroxypropyl cellulose, attapulgite, bentonite, hectorite, montmorillonite, silica gel, fumed silicon dioxide, colloidal silicon dioxide, acacia, agar, carrageenan, guar gum, locust bean gum, pectin, sodium alginate, propylene glycol alginate, tamarind gum, xanthan gum, carbomer, povidone, sodium starch glycolate, starches, tragacanth, magnesium aluminum silicate, aluminum silicate, magnesium silicate, gelatin, glycyrrhizin and the like. These suspending agents can further impart different flow properties to the suspension. The flow properties of the suspension can be Newtonian, plastic, pseudoplastic, thixotropic or combinations thereof. Mixtures of suspending agents may also be used to optimize flow properties and viscosity.

Suitable buffering agents may include, but are not limited to, one or more of a bicarbonate salt of a Group IA metal, an alkali earth metal buffering agent, amino acids, an acid salt of an amino acid, an alkali salt of an amino acid, and combinations of any of the foregoing.

Moreover, the composition of the invention optionally include usual auxiliaries known in the art such as saliva stimulating agents like citric acid, lactic acid, malic acid, succinic acid, ascorbic acid, adipic acid, fumaric acid, tartaric acids; cooling sensation agents like maltitol, monomenthyl succinate, ultracool; stabilizers like gums, agar; taste masking agents like acrylic polymers, copolymers of acrylates, celluloses, resins; coloring agents like titanium dioxide, natural food colors, dyes suitable for food, drug and cosmetic applications; preservatives like alpha-tocopherol, citric acid, butylated hydroxytoluene, butylated hydroxyanisole, ascorbic acid, fumaric acid, malic acid, sodium ascorbate or ascorbic acid palmitate or effervescing agents like citric acid, tartaric acid, sodium bicarbonate, sodium carbonate and the like.

The dosage form of the present invention may be in form of a tablet, a capsule, granules, a tablet in tablet, an orally disintegrating tablet, pellets, tablet/s in capsule, granules/pellets in capsule, a bilayer tablet, a trilayer tablet, an in-lay tablet or suspension.

The tablet-in-tablet dosage form of the invention may be prepared by compressing metoprolol with one or more rate controlling excipient, to form a core tablet; and compressing one or more anti-platelet agent optionally along with one or more pharmaceutically acceptable excipient onto the said core tablet to form a compressed outer tablet.

The tablet-in-tablet dosage form of the invention may be prepared by compressing metoprolol with one or more rate controlling excipient to form a core tablet; and compressing one or more anti-platelet agents optionally along with one or more pharmaceutically acceptable excipients onto said core tablet to form a compressed outer tablet.

In an embodiment, the tablet-in-tablet dosage form is be prepared by blending metoprolol with rate controlling excipient and other pharmaceutically acceptable excipients. The prepared blend was compressed to form a core tablet. Separately, anti-platelet agent is blended with one or more pharmaceutically acceptable excipients. Some portion of the above blend is placed in die and the core tablet was placed in center of the blend, the remaining blend is filled in die and compressed such that the metoprolol tablet forms inner tablet and anti-platelet agent forms outer tablet.

The once-a-day composition of the dosage form may include a tablet comprising an extended release metoprolol with one or more rate controlling excipient, wherein the tablet is inlayed in another layer comprising anti-platelet agent and optionally other pharmaceutically acceptable excipients.

In another embodiment, the once-a-day dosage form is prepared by blending metoprolol with rate controlling excipient and one or more other pharmaceutically acceptable excipients. The prepared blend is compressed to form tablets. The formed tablets then coated with dispersion comprising an anti-platelet agent, dissolved or dispersed in suitable solvent system along with one or more pharmaceutically acceptable excipient. The outer coating may completely or partially surround the metoprolol tablet.

In another embodiment, the once-a-day dosage form may be prepared by blending two portions with one or more pharmaceutically acceptable excipients followed by compression. First portion may be prepared by coating the inert core with a solution or suspension of metoprolol in a solvent. The metoprolol drug layer is further coated with one or more release-controlling layers. Second portion may be prepared by coating one or more anti-platelet agent on an inert core, optionally along with one or more rate controlling layers.

In another embodiment, the once-a-day dosage form may be prepared by blending two portions with one or more pharmaceutically acceptable excipients followed by compression. The first portion was prepared by coating the inert core with a dispersion comprising metoprolol, one or more rate controlling excipients in a solvent. The coated inner core can further be coated with one or more rate controlling layers or seal coat. The second portion was prepared by coating the inert core with a dispersion comprising an anti-platelet agent in a solvent.

In an embodiment, the once-a-day dosage form may include a tablet comprising an extended release metoprolol with one or more rate controlling excipient, wherein the tablet is inlayed in another layer comprising an anti-platelet agent and optionally other pharmaceutically acceptable excipients.

In a further embodiment, the inlayed dosage form can be prepared by blending metoprolol with rate controlling excipient and other pharmaceutically acceptable excipients. The prepared blend was compressed to form a core tablet. One or more anti-platelet agents are separately blended with one or more pharmaceutically acceptable excipients. Some portion of the above blend was placed in die and the core tablet was placed in a way such that the upper surface of metoprolol tablet is completely exposed after compression.

In a further embodiment, the once-a-day dosage form may be prepared by compressing a first layer comprising an extended release metoprolol along with one or more rate controlling excipients and a second layer comprising one or more anti-platelet agents, one or more pharmaceutically acceptable excipients and, optionally with rate controlling excipient into a bi-layer tablet.

In a further embodiment, the bi-layer dosage form is prepared by blending metoprolol with rate controlling excipient and other pharmaceutically acceptable excipients. The prepared blend was compressed to form a first layer. Onto this first layer a blend comprising anti-platelet agent with one or more pharmaceutically acceptable excipients is compressed to form a bi-layer tablet.

The present invention further provides a method of treating one or more disorders selected form hypertension, congestive heart failure, angina, myocardial infarction, arteriosclerosis, diabetic nephropathy, diabetic cardiac myopathy, renal insufficiency, peripheral vascular disease, left ventricular hypertrophy, cognitive dysfunction, and chronic heart failure, wherein the method comprises administering a pharmaceutical dosage form of the present invention to a patient in need of such treatment.

In another aspect, the present invention provides a method of treating hypertension, wherein the method comprises administering a pharmaceutical dosage form of the present invention to a patient in need of such treatment.

In an embodiment, a method of treating congestive heart failure comprises administering a pharmaceutical dosage form of the present invention to a patient in need of such treatment.

In another embodiment, a method of myocardial infarction comprises administering a pharmaceutical dosage form of the present invention to a patient in need of such treatment.

The invention is further illustrated by the following examples which are provided merely to be exemplary of the invention and do not limit the scope of the invention. Certain modifications and equivalents will be apparent to those skilled in the art and are intended to be included within the scope of the invention.

### Example 1

### Metoprolol Succinate ER/Clopidogrel Bisulfate Tablet 50/75 mg

TABLE 1
Metoprolol ER and Clopidogrel Tablet

| Stage | Sub-stage | Ingredients | % |
|---|---|---|---|
| Metoprolol | Seal coat I | Micro crystalline cellulose spheres | 0.1-20 |
| Blend | | Ethyl cellulose | 0.01-20 |
| | | Triethyl citrate | 0.001-1 |
| | Drug Layering | Metoprolol Succinate | 2-70 |
| | (Metoprolol | Opadry Clear | 0.1-10 |
| | succinate) | | |
| | Extended | Ethyl cellulose | 0.1-30 |
| | Release coating-I | Opadry Clear | 0.1-10 |
| | Extended | Eudragit L30-D55 | 0.1-10 |
| | Release coating- | Triethyl citrate | 0.001-5 |
| | II | Talc | 0.1-10 |
| | Seal coat II | Opadry Clear | 0.1-10 |
| | PEG coating | Polyethylene glycol | 0.1-10 |
| | Blending | Silicified microcrystalline cellulose | 5-70 |
| | | Croscarmellose sodium | 1-25 |
| | | Polyethylene glycol | 0.1-25 |
| Clopidogrel | Addition of Drug | Clopidogrel Bisulfate | 5-30 |
| Blend | (Clopidogrel | Polyethylene glycol | 0.5-10 |
| | Bisulfate) | Microcrystalline cellulose | 5-40 |
| | | Mannitol | 0.1-25 |
| | | Hydroxypropyl Cellulose | 0.1-20 |
| | | Glyceryl Behenate | 0.01-5 |
| | | Hydrogenated castor oil | 0.01-5 |
| Lubrication & | Lubrication | Sodium Stearyl Fumarate | 0.001-5 |
| Compression | | | |
| Film coating | Film coating | Opadry | 0.1-10 |

### Procedure:

Preparation of Metoprolol Blend:

Microcrystalline cellulose spheres were given seal coat I of ethyl cellulose. These seal coated I pellets were subjected to metoprolol succinate layering with a binder in aqueous solvent system. Drug layered pellets were provided with Extended Release coating-I using Ethyl cellulose and opadry. An Extended Release coating-II of Eudragit was given using Plasticizer (triethyl citrate) & talc. Seal coat-II was given to Extended Release coated-II pellets followed by PEG coating in suitable solvent system. Final metoprolol blend was prepared by blending PEG coated pellets with Silicified microcrystalline cellulose, Croscarmellose sodium and Polyethylene glycol.

Preparation of Clopidogrel Blend:

Compacts of Clopidogrel bisulfate, PEG, Microcrystalline Cellulose, Mannitol and Hydroxypropyl cellulose were prepared in roller compactor followed by granules preparation from the same. Separately granules of Micro crystalline cellulose, Mannitol & Hydroxypropyl cellulose were prepared by wet granulation method. Both the blends were lubricated with Glyceryl Behenate and Hydrogenated Castor Oil.

Lubrication & Compression:

Metoprolol blend and Clopidogrel blend were lubricated with Sodium Stearyl Fumarate or any other suitable lubricant. Lubricated blend was compressed into a tablet.

Coating:

An opadry coat was given to core tablets.

TABLE 2
Dissolution profile
Tablets obtained from example 1 were subjected to dissolution studies.
The results of dissolution studies performed are provided in Table 2.

| Dissolution of | | Dissolution of | |
|---|---|---|---|
| Metoprolol succinate | | Clopidogrel bisulfate | |
| Method: 500 ml of | | Method: 1000 ml of | |
| pH 6.8 phosphate buffer, | | pH 2.0 hydrochloric acid, | |
| USP II apparatus at 50 rpm | | USP II apparatus at 50 rpm | |
| Time points (h) | % Drug dissolved | Time points (min) | % Drug dissolved |
| 1 | 4 | 10 | 72 |
| 2 | 8 | 15 | 83 |
| 4 | 20 | 20 | 90 |
| 6 | 42 | 30 | 97 |
| 8 | 53 | 45 | 98 |
| 10 | 69 | 60 | 99 |
| 12 | 76 | — | — |
| 16 | 89 | — | — |
| 20 | 95 | — | — |
| 24 | 96 | — | — |

### Example 2

### Metoprolol Succinate ER/Clopidogrel Bisulfate/Aspirin Capsule 50/81/25 mg

TABLE 3
Metoprolol ER, Aspirin and Clopidogrel Capsule

| Stage | Sub-Stage | Ingredients | % |
|---|---|---|---|
| Metoprolol | Seal coat I | Micro crystalline cellulose spheres | 0.1-20 |
| pellets | | Ethyl cellulose | 0.01-20 |
| | | Triethyl citrate | 0.001-1 |
| | Drug Layering | Metoprolol succinate | 2-70 |
| | (Metoprolol | Opadry Clear | 0.1-10 |
| | succinate) | | |
| | Extended Release | Ethyl cellulose | 0.1-30 |
| | coating-I | Opadry Clear | 0.1-10 |
| | Extended Release | Eudragit L30-D55 | 0.1-10 |
| | coating-II | Triethyl citrate | 0.001-5 |
| | | Talc | 0.1-10 |
| | Seal coat II | Opadry Clear | 0.1-10 |
| | PEG coating | Polyethylene glycol | 0.1-10 |
| Aspirin | Drug Layering | Micro crystalline cellulose spheres | 0.1-20 |
| pellets | (Aspirin) | Opadry clear | 0.01-10 |
| | | Aspirin | 0.1-20 |
| | Seal coat-I | Opadry | 0.001-5 |
| | Seal coat-II | Mannitol | 0.001-5 |
| | PVA coating | Polyvinyl alcohol based opadry | 1-25 |
| Clopidogrel | Addition of | Clopidogrel Bisulfate | 5-30 |
| blend | Clopidogrel Bisulfate | Polyethylene glycol | 0.5-10 |
| | | Microcrystalline cellulose | 5-40 |
| | | Mannitol | 0.1-25 |
| | | Hydroxypropyl Cellulose | 0.1-20 |
| | | Glyceryl Behenate | 0.01-5 |
| | | Hydrogenated castor oil | 0.01-5 |
| Lubrication & | Lubrication | Sodium Stearyl Fumarate | 0.001-5 |
| capsulation | | | |

### Procedure:

Preparation of Metoprolol Pellets:

Microcrystalline cellulose spheres were given seal coat of ethyl cellulose. These seal coated pellets were subjected to metoprolol succinate layering with a binder in aqueous solvent system. Drug layered pellets were provided with Extended Release coating-I using Ethyl cellulose and opadry. An Extended Release coating-II of Eudragit was given using Plasticizer (triethyl citrate) & talc. The extended release pellets were then seal coated Seal coat-II was given to Extended Release coated-II pellets followed by PEG coating in suitable solvent system.

Preparation of Aspirin Pellets:

Microcrystalline cellulose spheres (or any other suitable spheres) were given with drug layering of aspirin using opadry as binder. Drug layered pellets were coated with seal coating-I and seal coating-II using opadry and Mannitol respectively. Seal coated-II pellets were further coated with PVA based opadry.

Preparation of Clopidogrel Blend:

Compacts of Clopidogrel bisulfate, PEG, Microcrystalline Cellulose, Mannitol and Hydroxypropyl Cellulose were prepared in roller compactor followed by granules preparation from the same. Separately granules of Micro crystalline cellulose, Mannitol & Hydroxypropyl Cellulose were prepared by wet granulation method. Both the blend were lubricated with Glyceryl Behenate and Hydrogenated Castor Oil.

Lubrication:

Metoprolol and aspirin pellets were lubricated with Sodium Stearyl Fumarate.

Capsulation:

Lubricated blend of Clopidogrel and lubricated pellets were filled into capsule shell of suitable size.

TABLE 4
Dissolution profile
Tablets obtained from example 2 were subjected to dissolution studies.
The results of dissolution studies performed are provided in Table 4.

| Dissolution of | | | | Dissolution of | |
|---|---|---|---|---|---|
| Metoprolol | | Dissolution of | | Clopidogrel | |
| succinate | | Aspirin | | Bisulfate | |
| Method: | | Method: | | Method: | |
| 500 ml of pH | | 900 ml of pH | | 1000 ml of pH | |
| 6.8 phosphate, | | 0.01N hydrochloric | | 2.0 hydrochloric | |
| buffer USP II | | acid, USP I | | acid, USP II | |
| apparatus at 50 rpm | | apparatus at 100 rpm | | apparatus at 50 rpm | |
| Time | % Drug | Time | % Drug | Time | % Drug |
| points (h) | dissolved | points (h) | dissolved | points (min) | dissolved |
| 1 | 3 | 10 | 43 | 10 | 78 |
| 2 | 7 | 15 | 63 | 15 | 89 |
| 4 | 21 | 20 | 83 | 20 | 96 |
| 6 | 40 | 30 | 99 | 30 | 97 |
| 8 | 51 | 45 | 101 | 45 | 99 |
| 10 | 67 | 60 | 101 | 60 | 99 |
| 12 | 75 | — | — | — | — |
| 16 | 88 | — | — | — | — |
| 20 | 94 | — | — | — | — |
| 24 | 95 | — | — | — | — |

### Example 3

### Metoprolol succinate ER/Clopidogrel Bisulfate/Aspirin Capsule 100/75/50 mg

TABLE 5
Metoprolol ER, Aspirin and Clopidogrel Capsule

| Stage | Sub-Stage | Ingredients | % |
|---|---|---|---|
| Metoprolol | Seal coat I | Micro crystalline cellulose spheres | 0.1-20 |
| pellets | | Ethyl cellulose | 0.01-20 |
| | | Triethyl citrate | 0.001-1 |
| | Drug Layering | Metoprolol succinate | 2-70 |
| | (Metoprolol | Opadry Clear | 0.1-10 |
| | succinate) | | |
| | Extended Release | Ethyl cellulose | 0.1-30 |
| | coating-I | Opadry Clear | 0.1-10 |
| | Extended Release | Eudragit L30-D55 | 0.1-10 |
| | coating-II | Triethyl citrate | 0.001-5 |
| | | Talc | 0.1-10 |
| | Seal coat II | Opadry Clear | 0.1-10 |
| | PEG coating | Polyethylene glycol | 0.1-10 |
| Aspirin pellets | Drug Layering | Micro crystalline cellulose spheres | 0.1-20 |
| | (Aspirin) | Opadry clear | 0.01-10 |
| | | Aspirin | 0.1-20 |
| | Seal coat-I | Opadry | 0.001-5 |
| | Seal coat-II | Mannitol | 0.001-5 |
| | PVA coating | Polyvinyl alcohol based opadry | 1-25 |
| Clopidogrel | Addition of | Clopidogrel Bisulfate | 5-30 |
| blend | Clopidogrel Bisulfate | Polyethylene glycol | 0.5-10 |
| | | Microcrystalline cellulose | 5-40 |
| | | Mannitol | 0.1-25 |
| | | Hydroxypropyl Cellulose | 0.1-20 |
| | | Glyceryl Behenate | 0.01-5 |
| | | Hydrogenated castor oil | 0.01-5 |
| Lubrication & | Lubrication | Sodium Stearyl Fumarate | 0.001-5 |
| capsulation | | | |

### Procedure:

Preparation of Metoprolol Pellets:

Microcrystalline cellulose spheres were given seal coat I of ethyl cellulose. These seal coated I pellets were subjected to metoprolol succinate layering with a binder in aqueous solvent system. Drug layered pellets were provided with Extended Release coating-I using Ethyl cellulose and opadry. An Extended Release coating-II of Eudragit was given using Plasticizer (triethyl citrate) & talc. Seal coat-II was given to Extended Release coated-II pellets followed by PEG coating in suitable solvent system.

Preparation of Aspirin Pellets:

Microcrystalline cellulose spheres (or any other suitable spheres) were given with drug layering of aspirin using opadry as binder. Drug layered pellets were coated with seal coating-I and seal coating-II using opadry and Mannitol respectively. Seal coated-II pellets were further coated with PVA based opadry.

Preparation of Clopidogrel Blend:

Compacts of Clopidogrel bisulfate, PEG, Microcrystalline Cellulose, Mannitol and Hydroxypropyl Cellulose were prepared in roller compactor followed by granules preparation from the same. Separately granules of Micro crystalline cellulose, Mannitol & Hydroxypropyl Cellulose were prepared by wet granulation method. Both the blend were lubricated with Glyceryl Behenate and Hydrogenated Castor Oil.

Lubrication:

Metoprolol and aspirin pellets were lubricated with Sodium Stearyl Fumarate.

Capsulation:

Lubricated blend of Clopidogrel and lubricated pellets were filled into capsule shell of suitable size.

TABLE 6
Dissolution profile
Tablets obtained from example 3 were subjected to dissolution studies.
The results of dissolution studies performed are provided in Table 6.

| Dissolution of | | | | Dissolution of | |
|---|---|---|---|---|---|
| Metoprolol | | Dissolution of | | Clopidogrel | |
| succinate | | Aspirin | | Bisulfate | |
| Method: | | Method: | | Method: | |
| 500 ml of pH | | 900 ml of pH | | 1000 ml of pH | |
| 6.8 phosphate, | | 0.01N hydrochloric | | 2.0 hydrochloric | |
| buffer USP II | | acid, USP I | | acid, USP II | |
| apparatus at 50 rpm | | apparatus at 100 rpm | | apparatus at 50 rpm | |
| Time | % Drug | Time | % Drug | Time | % Drug |
| points (h) | dissolved | points (h) | dissolved | points (min) | dissolved |
| 1 | 4 | 10 | 41 | 10 | 76 |
| 2 | 9 | 15 | 60 | 15 | 86 |
| 4 | 22 | 20 | 79 | 20 | 92 |
| 6 | 42 | 30 | 95 | 30 | 98 |
| 8 | 53 | 45 | 98 | 45 | 98 |
| 10 | 70 | 60 | 100 | 60 | 98 |
| 12 | 76 | — | — | — | — |
| 16 | 90 | — | — | — | — |
| 20 | 98 | — | — | — | — |
| 24 | 98 | — | — | — | — |

### Example 4

### Metoprolol succinate ER/Ticagrelor Tablet 50/90 mg

TABLE 7
Metoprolol succinate ER and Ticagrelor tablet

| Stage | Stage | Ingredients | % |
|---|---|---|---|
| Metoprolol | Seal coat I | Micro crystalline | 0.1-20 |
| Pellets | | cellulose spheres | |
| | | Ethyl cellulose | 0.01-20 |
| | | Triethyl citrate | 0.001-1 |
| | Drug Layering | Metoprolol succinate | 2-40 |
| | (Metoprolol | Opadry Clear | 0.1-10 |
| | succinate) | | |
| | Extended Release | Ethyl cellulose | 0.1-30 |
| | coating-I | Opadry Clear | 0.1-10 |
| | Extended Release | Eudragit L30-D55 | 0.1-10 |
| | coating-II | Triethyl citrate | 0.001-5 |
| | | Talc | 0.1-10 |
| | Seal coat II | Opadry Clear | 0.1-10 |
| | PEG coating | Polyethylene glycol | 0.1-10 |
| | Blending | Polyethylene glycol | 0.1-10 |
| | | Prosolv SMCC 90 | 5-50 |
| | | Croscarmellose sodium | 0.1-10 |
| Ticagrelor | Addition of Drug | Ticagrelor | 5-70 |
| blend | (Ticagrelor) | Mannitol | 10-80 |
| | | Dibasic calcium | 0.5-15 |
| | | phosphate | |
| | | Hydroxypropyl cellulose | 0.1-10 |
| | | Sodium starch glycolate | 0.1-10 |
| Blending & | Blending and | Sodium Stearyl Fumarate | 0.01-5 |
| Lubrication | Lubrication | | |
| Film coating | Film coating | Opadry white | 0.1-10 |

### Procedure:

Microcrystalline cellulose spheres was given seal coat I of ethyl cellulose. These seal coated I pellets were subjected to metoprolol succinate layering with a binder in aqueous solvent system. Drug layered pellets were provided with Extended Release coating-I using Ethyl cellulose and opadry. An Extended Release coating-II of eudragit was given using plasticizer, triethyl citrate & talc. Seal coat-II was given to Extended Release coated-II pellets followed by PEG coating in suitable solvent system. A blend from PEG coated pellets was prepared by blending it with polyethylene glycol, prosolv SMCC 90 and croscarmellose sodium.

Granules of Ticagrelor were prepared separately by wet granulation. Ticagrelor was mixed with mannitol, dibasic calcium phosphate and sodium starch glycolate, which were granulated using solution of hydroxypropyl cellulose in water. Prepared granules were dried and mill to suitable size and mixed with disintegrant. Prepared ticagrelor blend was mixed with metoprolol blend and lubricated with sodium stearyl fumarate. Prepared blend was compressed into tablet. An opadry coat was given to core tablets.

### Example 5

### Clinical Study on Human being

The study method involved a multicenter, randomized, placebo-controlled, unbalanced factorial study for lowering the blood pressure and assessing the risk of stroke. Patients, with confirmed diagnosis of stage II hypertension were eligible to participate in the study. Patients were randomized to one of many treatment groups: Group I were administered extended-release metoprolol succinate (Eq 25 mg tartrate, Eq 50 mg tartrate, Eq 100 mg tartrate), Group II were administered clopidogrel (75 mg), Group III were administered extended release of metoprolol succinate/Immediate release clopidogrel (dosages of present invention). After one month of therapy non-responder patients were managed with dose-titration or rescue medication.

Treatment groups were well balanced at base line and achieved absolute change at one week from the baseline in blood pressure. Inventors of the present application surprisingly found at least 10% improvement in blood pressure (systolic blood pressure and diastolic blood pressure) after 3 months treatment and at least 5% reduction in the risk of stoke after 6 months of treatment.

While the present invention has been described in terms of its specific embodiments, certain modifications and equivalents will be apparent to those skilled in the art and are intended to be included within the scope of the present invention.

We claim:

1. A pharmaceutical dosage form for treatment of cardiovascular disorders suitable for once daily administration comprising a fixed dose combination of metoprolol in extended release form and one or more anti-platelet agents along with one or more rate controlling excipients.

2. The pharmaceutical dosage form of claim 1, wherein the antiplatelet agent comprises one or more of clopidogrel, aspirin, prasugrel, ticagrelor, ticlopidine, anagrelide, cilostazol, and dipyridamole.

3. The pharmaceutical dosage form of claim 1, wherein the antiplatelet agent comprises one or more of clopidogrel, ticagrelor, and aspirin.

4. The pharmaceutical dosage form of claim 1, wherein anti-platelet agent is clopidogrel and/or aspirin.

5. The pharmaceutical dosage form of claim 1, wherein the dosage form comprises about 25 mg to about 200 mg of metoprolol and about 5 mg to about 770 mg of antiplatelet agent.

6. The pharmaceutical dosage form of claim 1, wherein the dosage form exhibits immediate release of anti-platelet agent.

7. The pharmaceutical dosage form of claim 1, wherein the dosage form comprises one or more water swellable or water insoluble inert cores coated with one or more rate controlling excipients.

8. The pharmaceutical dosage form of claim 7, wherein the water swellable core comprises microcrystalline cellulose, hydroxypropyl methylcellulose, starch, or mixtures thereof.

9. The pharmaceutical dosage form of claim 6, wherein the water insoluble inert core comprises silicon dioxide, glass particles, plastic resin particles, or mixtures thereof.

10. The pharmaceutical dosage form of claim 1, wherein the rate controlling excipient comprises polymeric rate controlling excipient, non-polymeric rate controlling excipient, or combination thereof.

11. The pharmaceutical dosage form of claim 10, wherein the polymeric and non-polymeric rate controlling excipient comprises one or more of cellulose derivatives; polyhydric alcohols; saccharides, gums and derivatives thereof; vinyl derivatives, polymers, copolymers or mixtures thereof; maleic acid copolymers; polyalkylene oxides or copolymers thereof; acrylic acid polymers and acrylic acid derivatives; fat; wax; fatty acid; fatty acid ester; long chain monohydric alcohol or their ester; or mixtures thereof.

12. The pharmaceutical dosage form of claim 1, wherein the dosage form exhibits a dissolution profile such that less than about 6% of metoprolol is released within 1 hour; between about 30% to about 50% of metoprolol is released within 6 hours and at least 90% of metoprolol is released after 20 hours when the release rate is measured in USP Type 2 Dissolution Apparatus (paddle, 50 rpm) using 500 ml of pH 6.8 phosphate buffer at 37° C.±0.5° C. as dissolution medium.

13. The pharmaceutical dosage form of claim 1, wherein the dosage form is in the form of a tablet, a capsule, granules, pellets, a tablet in tablet, tablet/s in capsule, granules or pellets in capsule, a bi-layer tablet, a trilayer tablet, and an in-lay tablet.

14. A method of treating a disorder selected from one or more of hypertension, congestive heart failure, angina, myocardial infarction, arteriosclerosis, diabetic nephropathy, diabetic cardiac myopathy, renal insufficiency, peripheral vascular disease, left ventricular hypertrophy, cognitive dysfunction, chronic heart failure, wherein the method comprises administering a pharmaceutical dosage form of claim 1 to a patient in need of such treatment.
